SEQUENCE LISTING (16 sequences)

SEQ ID NO: 1 - protein, 459 aa, Oryctolagus cuniculus
METGLRWLLLVAVLKGVQCQSVEESGGRLVTPGTPLTLTCTVSGFSLSNYAMSWVRQAPGKGLEYIGYINTDGSAYYASWTKGRFTISKTSTTVDLKITSPTTEDTATYFCARGWASNSIYNLKLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 2 - protein, 236 aa, Oryctolagus cuniculus
MDTRAPTQLLGLLLLWLPGARCAYDMTQTPPSVSAAVGGTVTIKCQASHNIYNNLAWYQQKPGQRPKLLIYGTSTLESGVPSRFRGSGSGTEYTLTISDLECADAATYYCQQSYLSNNIENVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 3 - protein, 458 aa, Oryctolagus cuniculus
METGLRWLLLVAVLKGVQCQSVEESGGRLVTPGTPLTLTCTVSGIDLSDYAMGWVRQAPGKGLEYIGIISSSGNTHYARWARGRFTISKTSSTTVDLKMTSLTTEDTATYFCARNYPGYANYALWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 4 - protein, 237 aa, Oryctolagus cuniculus
MDTRAPTQLLGLLLLWLPGATFARIVMTQTPASVSAAVGGTVTIKCQASESISNWLSWYQQKPGQPPKLLIYRASTLASGVPSRFSGSGSGTEYTLTISDLECADAATYYCQQDYIYNDIDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 5 - protein, 5 aa, Oryctolagus cuniculus
NYAMS

SEQ ID NO: 6 - protein, 16 aa, Oryctolagus cuniculus
YINTDGSAYYASWTKG

SEQ ID NO: 7 - protein, 12 aa, Oryctolagus cuniculus
GWASNSIYNLKL

SEQ ID NO: 8 - protein, 11 aa, Oryctolagus cuniculus
QASHNIYNNLA

SEQ ID NO: 9 - protein, 7 aa, Oryctolagus cuniculus
GTSTLES

SEQ ID NO: 10 - protein, 12 aa, Oryctolagus cuniculus
QQSYLSNNIENV

SEQ ID NO: 11 - protein, 5 aa, Oryctolagus cuniculus
DYAMG

SEQ ID NO: 12 - protein, 16 aa, Oryctolagus cuniculus
IISSSGNTHYARWARG

SEQ ID NO: 13 - protein, 10 aa, Oryctolagus cuniculus
NYPGYANYAL

SEQ ID NO: 14 - protein, 11 aa, Oryctolagus cuniculus
QASESISNWLS

SEQ ID NO: 15 - protein, 7 aa, Oryctolagus cuniculus
RASTLAS

SEQ ID NO: 16 - protein, 12 aa, Oryctolagus cuniculus
QQDYIYNDIDNA

(19) United States
(12) Patent Application Publication
(10) Pub. No.: US 2025/0180565 A1
(43) Pub. Date: Jun. 5, 2025

(54) ANTI-HER2/NEU ANTIBODIES AND METHODS OF USE

(71) Applicant: Martell Diagnostic Laboratories, Inc., Roseville, MN (US)

(72) Inventors: Franklin Pass, Edina, MN (US); Steven Stoesz, Minneapolis, MN (US); Justin Lengfeld, Brooklyn Park, MN (US)

(21) Appl. No.: 18/837,130

(22) PCT Filed: Feb. 9, 2023
(86) PCT No.: PCT/US23/62260

§ 371 Date: Aug. 8, 2024

Related U.S. Application Data
(60) Provisional application No. 63/267,755, filed on Feb. 9, 2022.

(51) Int. Cl. G01N 33/574; C07K 16/32; G01N 33/573

(52) U.S. Cl. CPC G01N 33/57488; C07K 16/32; G01N 33/573; C07K 2317/33; G01N 2333/912

(57) ABSTRACT

Provided herein are anti-HER2 monoclonal antibodies or binding fragments thereof and uses thereof.

### PRIORITY

This patent application claims the benefit of priority to U.S. Provisional Application Ser. No. 63/267,755, filed Feb. 9, 2022, which is incorporated by reference herein in its entirety.

### INCORPORATION BY REFERENCE OF SEQUENCE LISTING

A Sequence Listing is provided herewith as an xml file, “2308531.xml” created on Feb. 8, 2023, and having a size of 16,325 bytes. The content of the xml file is incorporated by reference herein in its entirety.

### BACKGROUND

Breast cancer treatment has improved considerably over the past 20 years as a result of, for example, earlier detection, better surgical techniques, a variety of new drugs, and new imaging methods (e.g., to detect recurrence).

HER2/neu is a member of the human epidermal growth factor receptor (HER/EGFR/ERBB) family. Amplification or over-expression of this oncogene has been shown to play a role in the development and progression of certain aggressive types of breast cancer. The protein has become a biomarker and target of therapy for approximately 30% of breast cancer patients.

The HER2/neu protein is proteolytically cleaved by membrane-associated serine proteases to release the extracellular domain, which then can be detected and measured in bodily fluids. A HER2/neu in vitro diagnostic (IVD) immunoassay for blood was introduced (as described in, for example, Carney et al., 2003, Clin. Chem., 49 (10): 1579-98), but the results were often confusing, and the test fell into clinical disuse.

### SUMMARY

Provided herein is an anti-HER2 monoclonal antibody or binding fragment thereof comprising a heavy chain variable domain (VH) comprising the amino acid sequence of SEQ ID NO: 1 or at least 95% identity thereto and a light chain variable domain (VL) comprising the amino acid sequence of SEQ ID NO:2 or at least 95% identity thereto. One aspect provides an anti-HER2 monoclonal antibody or binding fragment thereof comprising a heavy chain and a light chain, wherein: (i) the heavy chain comprises three CDR regions having the amino acid sequence SEQ ID NO: 5, 6, 7 or at least 95% identity thereto; and (ii) the light chain comprises three CDR regions having the amino acid sequence SEQ ID NO: 8, 9, 10 or at least 95% identity thereto. Another aspect provides anti-HER2 monoclonal antibody or binding fragment thereof as described herein wherein the antibody is conjugated to a detection agent. One aspect provides a composition comprising the anti-HER2 antibody as described herein and a carrier.

One aspect provides a method to detect HER2 polypeptides or fragments thereof in a test sample comprising: (a) contacting the anti-HER2 monoclonal antibody or binding fragment thereof described herein with a test sample under conditions that allow polypeptide/antibody complexes to form; and (b) detecting polypeptide/antibody complexes of a), wherein the detection of polypeptide/antibody complexes is an indication that the HER2 polypeptide is present in the sample. Another method provides a way to monitor HER2 polypeptides or fragments thereof in a sample from a subject comprising: (a) contacting at least one of the anti-HER2 monoclonal antibodies or binding fragments thereof described herein with said sample under conditions that allow polypeptide/antibody complexes to form; (b) detecting polypeptide/antibody complexes of a), wherein the detection of polypeptide/antibody complexes indicates HER2 polypeptides or fragments thereof are present in the subject; and (c) performing steps (a) and (b) at a plurality of time points so as monitor HER2 polypeptides or fragments thereof in said subject over time. In one embodiment the method further comprises contacting the sample of (a) with a second anti-HER2 antibody or fragment thereof comprising a heavy chain variable domain (VH) comprising the amino acid sequence of SEQ ID NO: 3 or at least 95% identity, a light chain variable domain (VL) comprising the amino acid sequence of SEQ ID NO: 4 or at least 95% identity or an anti-HER2 antibody or fragment thereof comprising a heavy chain and a light chain, wherein (i) the heavy chain comprises three CDR regions having the amino acid sequence SEQ ID NO: 11, 12, 13 or at least 95% identity; and (ii) the light chain comprises three CDR regions having the amino acid sequence SEQ ID NO: 14, 15, 16 or at least 95% identity. In one embodiment, the sample is contacted in a) with: (i) a capture antibody or a binding fragment thereof, and (ii) a detection antibody or a binding fragment thereof. In one embodiment, the capture and detection antibodies bind to HER2 or a polypeptide thereof. In one embodiment, the capture antibody is immobilized. In another embodiment, the detection antibody comprises a detection agent.

In one embodiment, the subject is being treated with a therapeutic agent. In one embodiment, the therapeutic agent is trastuzumab, trastuzumab emtansine, pembrolizumab, pertuzumab, nivolumab, atezolizumab or a combination thereof. In another embodiment, the subject is being treated with trastuzumab, trastuzumab emtansine, pembrolizumab, pertuzumab, nivolumab, atezolizumab or a combination thereof, wherein the trastuzumab, trastuzumab emtansine, pembrolizumab, pertuzumab, nivolumab, atezolizumab or a combination thereof does not interfere or only moderately interferes with the binding of the capture and/or detection antibodies or binding fragments thereof. In one embodiment the subject is ou can be treated for cancer, such treatment including small molecules, immunotherapy, surgical, chemotherapy and/or radiation treatment. In one embodiment, the sample is lymph node or tissue aspirate (e.g., breast), serum, whole blood, plasma, urine, saliva, tears, cerebrospinal fluid, supernatant from normal cell lysates, supernatant from pre-neoplastic cell lysates, supernatant from neoplastic cell lysates and/or supernatants from carcinoma cell lines maintained in tissue culture.

In one embodiment, the detection in b) is carried out with the use of a lateral flow assay.

### DETAILED DESCRIPTION OF THE INVENTION

The practice of the methods and compositions described herein may employ, unless otherwise indicated, conventional techniques of pharmaceutical chemistry, molecular biology, drug formulation techniques, dosage regimes, immunology and biochemistry, all of which are within the skill of those who practice in the art.

Provided herein are recombinant rabbit monoclonal antibodies which bind with specificity to HER2, such as to the extracellular domain of HER2 (e.g., human HER2 receptor). The antibodies described herein provide several improvements over other anti-HER2 antibodies, in addition to their binding specificity, they exhibit little to no interference with therapeutic agents, making an immunoassay based on the antibodies provided herein more valuable in terms of providing much needed accurate information to the doctor/patient.

Substances that alter the measurable concentration of the analyte or alter antibody binding can potentially result in immunoassay interference. Interfering substances may lead to falsely elevated or falsely low analyte concentration in one or more assay systems depending on the site of the interference in the reaction. Interference in an immunoassay may lead to the misinterpretation of a patient's results by the laboratory and the wrong course of treatment being given by the physician. For example, pertuzumab is one of the most common therapies used in the treatment of breast cancer to lower the level of HER2 in serum; unfortunately, pertuzumab interferes with many of the diagnostic assays that are currently used in the clinic, which drastically reduces the reliability of those assays. Interference of a diagnostic assay by a therapeutic antibody can take place at any number of stages of the assay or with any of the components involved in the assay (e.g., the capture antibody and/or the detection antibody). Significantly, pertuzumab, trastuzumab, margetuximab, and/or HER2 small molecule inhibitors (e.g., lapatinib, neratinib) exhibit little to no interference with the antibodies/immunoassay described herein, which allows for greater accuracy when testing and/or monitoring patients that are being administered a therapeutic regimen for HER2 positive breast cancer.

### Definitions

For the purposes of clarity and a concise description, features can be described herein as part of the same or separate embodiments; however, it will be appreciated that the scope of the invention may include embodiments having combinations of all or some of the features described.

The terminology used herein is for the purpose of describing particular embodiments only and is not intended to be limiting of the invention. Unless defined otherwise, all technical and scientific terms used herein have the same meaning as commonly understood by one of ordinary skill in the art to which this invention belongs. The following definitions are intended to aid the reader in understanding the present invention but are not intended to vary or otherwise limit the meaning of such terms unless specifically indicated.

As used herein, the indefinite articles “a”, “an” and “the” should be understood to include plural reference unless the context clearly indicates otherwise. Thus, for example, reference to “an inhibitor” refers to one or more agents with the ability to inhibit a target molecule, and reference to “the method” includes reference to equivalent steps and methods known to those skilled in the art, and so forth.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the term “about” means plus or minus 10% of the indicated value. For example, about 100 means from 90 to 110.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

Transmembrane protein HER2 (human epidermal growth factor receptor 2) or HER2/neu is also known as receptor tyrosine-protein kinase erbB-2, CD340 (cluster of differentiation 340), proto-oncogene Neu, Erbb2 (rodent), or ERBB2 (human); a protein that in humans is encoded by the ERBB2 (erythroblastic oncogene B) gene. The HER2 protein has a molecular weight of about 185 kiloDaltons (kDa) and consists of an intracellular tyrosine kinase domain, a transmembrane domain and an extracellular domain.

Amplification, also known as the over-expression of the ERBB2 gene, occurs in approximately 15-30% of breast cancers, also known as HER2 positive breast cancer. HER2-positive breast cancer is a breast cancer that tests positive for a protein called human epidermal growth factor receptor 2 (HER2). This protein promotes the growth of cancer cells.

In about 1 of every 5 breast cancers, the cancer cells have extra copies of the gene that makes the HER2 protein. HER2-positive breast cancers tend to be more aggressive than other types of breast cancer. It is associated with increased disease recurrence and a poor prognosis; however, drug agents targeting HER2 in breast cancer have significantly positively altered the otherwise poor-prognosis natural history of HER2-positive breast cancer. It is recommended that every invasive breast cancer be tested for the presence of HER2 because the results significantly impact treatment recommendations and decisions.

As used herein, “detecting” refers to the action or process of identifying the presence of that which is being detected, such as HER2/neu in a sample. As used herein, the term “sample” is defined as blood, serum, plasma, urine, saliva, tears, cerebrospinal fluid, supernatant from normal cell lysates, supernatant from pre-neoplastic cell lysates, supernatant from neoplastic cell lysates, supernatants from carcinoma cell lines maintained in tissue culture, and breast aspirates or biopsies. Thus, any number of biological samples can be used in the immunoassays described herein including, without limitation, blood, serum, plasma, urine, saliva, tears, cerebrospinal fluid, supernatant from cell lysates (e.g., normal cells, pre-neoplastic cells, neoplastic cells, carcinoma cells), or breast aspirates or biopsies.

As used herein, “monitoring” refers to the action or process of identifying the presence of that which is being detected at least twice over a period of time.

The term “antibodies” refers to an intact antibody or an antigen-binding portion or fragment thereof that competes with the intact antibody for antigen binding. The term “antibodies” also includes any type of antibody molecule or specific binding molecule that specifically binds HER2. The terms “antigen-binding portion” of an antibody, “antigen-binding fragment” of an antibody, and the like, as used herein, include any naturally occurring, enzymatically obtainable, synthetic, or genetically engineered polypeptide, glycoprotein, or immunoglobulin that specifically binds to HER2 protein. Antigen-binding fragments of an antibody may be derived, e.g., from full antibody molecules using any suitable standard techniques such as proteolytic digestion or recombinant genetic engineering techniques involving the manipulation and expression of nucleic acids encoding antibody variable and optionally constant domains.

A monoclonal antibody is an antibody obtained from a group of substantially homogeneous antibodies. A group of substantially homogeneous antibodies can contain a small amount of mutants or variants. Monoclonal antibodies are highly specific and interact with a single antigenic site. Each monoclonal antibody typically targets a single epitope, while polyclonal antibody populations typically contain various antibodies that target a group of diverse epitopes. Monoclonal antibodies can be produced by many methods including, for example, hybridoma methods (Kohler and Milstein, Nature 256:495, 1975), recombination methods (U.S. Pat. No. 4,816,567), and isolation from phage antibody libraries (Clackson et al., Nature 352:624-628, 1991; Marks et al., J. Mol. Biol. 222:581-597, 1991).

The terms “subject,” “mammal” and “mammalian subject” as used herein refers to any animal classified as a mammal, including humans, higher non-human primates, rodents, and domestic and farm animals, such as cows, horses, dogs, and cats. In some embodiments of the invention, the mammal is a human (male or female).

As used herein, the terms “including”, “includes”, “having”, “has”, “with”, or variants thereof, are intended to be inclusive similar to the term “comprising.”

As used herein, said “contain”, “have” or “including” include “comprising”, “mainly consist of”, “basically consist of” and “formed of”; “primarily consist of”, “generally consist of” and “comprising of” belong to generic concept of “have” “include” or “contain”.

The terms “comprises,” “comprising,” and the like can have the meaning ascribed to them in U.S. Patent Law and can mean “includes,” “including” and the like. As used herein, “including” or “includes” or the like means including, without limitation.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### Rabbit Antibodies/Polypeptides

Rabbit monoclonal antibodies (e.g., anti-human HER2 antibodies) are a useful for many applications, including immunofluorescence, immunohistochemistry, flow cytometry, western blot, and ELISA assays. Compared to other animal models (e.g., mouse and rat), rabbits provide a better system for monoclonal antibody production because the rabbit immune system responds to a broader range of antigens. Also, physically, rabbits are larger animals with larger spleens that can produce more antibodies.

Rabbit monoclonal antibodies are similar to traditional mouse monoclonal antibodies while offering better specificity and sensitivity. Rabbits are immunized and the resulting spleen cells are fused with partner cells to make an immortal cell line that expresses antibodies. The antibodies are derived from a single clone and characterized for performance in applications. A clone or clones are then selected for antibody production.

As the rabbit natural repertoire is more diverse than the mouse, and the spleen is larger, their antibodies exhibit higher affinity for the antigen. Thus, rabbit monoclonal antibodies tend to give superior sensitivity in the application for which the clones were screened. An additional advantage of the rabbit diversity is that it allows for epitope recognition that may not be feasible with other systems. Other advantages include, natural diversity, high affinity and specificity, novel epitope recognition, cross-reactivity to human and mouse targets and ease of humanization. And, as provided herein, antibodies can be provided that show little to no interference with the therapeutic agents, such as other antibodies, peptides or small molecules.

A light or heavy chain variable region of an antibody has four framework regions interrupted by three hypervariable regions, known as complementary determining regions (CDRs). CDRs determine the specificity of antigen binding. The heavy chain and light chain each have three CDRs, designated from the N terminus as CDR1, CDR2, and CDR3 with the four framework regions flanking these CDRs. The amino acid sequences of the framework region are highly conserved and CDRs can be transplanted into other antibodies. Therefore, a recombinant antibody can be produced by combining CDRs from one or more antibodies with the framework of one or more other antibodies. Antibodies of the invention include antibodies that comprise at least one, two, three, four, five, or six (or combinations thereof) of the CDRs of any of the monoclonal antibodies described herein.

Polypeptides/antibodies of the invention comprise full-length rabbit anti-HER2/neu heavy chain variable regions, full-length rabbit light chain variable regions, binding fragments or variants thereof, and combinations thereof.

### 1C5 (Capture Antibody) Sequences:

Heavy chain (SEQ ID NO: 1 (below); CDR1, 2, and 3 are SEQ ID NO: 5,
6 and 7, respectively, provided in Table A).
(SEQ ID NO: 1)
METGLRWLLLVAVLKGVQCQSVEESGGRLVTPGTPLTLTCTVSGFSLSNYAMSWVRQ
APGKGLEYIGYINTDGSAYYASWTKGRFTISKTSTTVDLKITSPTTEDTATYFCARGW
ASNSIYNLKLWGPGTLVTVSSGQPKAPSVFPLAPCCGDTPSSTVTLGCLVKGYLPEPV
TVTWNSGTLTNGVRTFPSVRQSSGLYSLSSVVSVTSSSQPVTCNVAHPATNTKVDKTV
APSTCSKPMCPPPELPGGPSVFIFPPKPKDTLMISRTPEVTCVVVDVSQDDPEVQFTW
YINNEQVRTARPPLREQQFNSTIRVVSTLPIAHQDWLRGKEFKCKVHNKALPAPIEKTI
SKARGQPLEPKVYTMGPPREELSSRSVSLTCMINGFYPSDISVEWEKNGKAEDNYKT
TPTVLDSDGSYFLYSKLSVPTSEWQRGDVFTCSVMHEALHNHYTQKSISRSPGK
Light chain (SEQ ID NO: 2 (below); CDR1, 2, and 3 are SEQ ID NO: 8,
9 and 10, respectively, provided in Table B).
(SEQ ID NO: 2)
MDTRAPTQLLGLLLLWLPGARCAYDMTQTPPSVSAAVGGTVTIKCQASHNIYNNLA
WYQQKPGQRPKLLIYGTSTLESGVPSRFRGSGSGTEYTLTISDLECADAATYYCQQSY
LSNNIENVFGGGTEVVVKGDPVAPTVLIFPPAADQVATGTVTIVCVANKYFPDVTVTW
EVDGTTQTTGIENSKTPQNSADCTYNLSSTLTLTSTQYNSHKEYTCKVTQGTTSVVQS
FNRGDC

TABLE A
Heavy Chain CDRs

| | CDR- | | | CDR- | | | CDR- | | |
|---|---|---|---|---|---|---|---|---|---|
| Clone | H1 | Residues | Length | H2 | Residues | Length | H3 | Residues | Length |
| 1C5 | 49- | NYAM | 5 | 68- | YINTDGSAYYAS | 16 | 114- | GWASNSIYN | 12 |
| | 53 | S (SEQ | | 83 | WTKG (SEQ ID | | 125 | LKL (SEQ ID | |
| | | ID | | | NO: 6) | | | NO: 7) | |
| | | NO: 5) | | | | | | | |

TABLE B
Light Chain CDRs

| | CDR- | | | CDR- | | | CDR- | | |
|---|---|---|---|---|---|---|---|---|---|
| Clone | L1 | Residues | Length | L2 | Residues | Length | L3 | Residues | Length |
| 1C5 | 46- | QASHNIYN | 11 | 72- | GTSTL | 7 | 111- | QQSYLSNNIE | 12 |
| | 56 | NLA (SEQ ID | | 78 | ES | | 122 | NV (SEQ ID | |
| | | NO: 8) | | | (SEQ | | | NO: 10) | |
| | | | | | ID | | | | |
| | | | | | NO: 9) | | | | |

1B7 (labeled antibody) sequences:
Heavy chain (SEQ ID NO: 3 (below); CDR1, 2, and 3 = SEQ ID NO: 11, 12, 13,
respectively, as provided in Table C):
(SEQ ID NO: 3)
METGLRWLLLVAVLKGVQCQSVEESGGRLVTPGTPLTLTCTVSGIDLSDYAMGWVRQ
APGKGLEYIGIISSSGNTHYARWARGRFTISKTSSTTVDLKMTSLTTEDTATYFCARNY
PGYANYALWGQGTLVTVSSGQPKAPSVFPLAPCCGDTPSSTVTLGCLVKGYLPEPVT
VTWNSGTLINGVRTFPSVRQSSGLYSLSSVVSVTSSSQPVTCNVAHPATNTKVDKTVA
PSTCSKPMCPPPELPGGPSVFIFPPKPKDTLMISRTPEVTCVVVDVSQDDPEVQFTWYI
NNEQVRTARPPLREQQFNSTIRVVSTLPIAHQDWLRGKEFKCKVHNKALPAPIEKTIS
KARGQPLEPKVYTMGPPREELSSRSVSLTCMINGFYPSDISVEWEKNGKAEDNYKTT
PTVLDSDGSYFLYSKLSVPTSEWQRGDVFTCSVMHEALHNHYTQKSISRSPGK
Light chain (SEQ ID NO: 4 (below0; CDR1, 2, and 3 = SEQ ID NO: 14, 15, 16
respectively, as provided in Table D):
(SEQ ID NO: 4)
MDTRAPTQLLGLLLLWLPGATFARIVMTQTPASVSAAVGGTVTIKCQASESISNWLS
WYQQKPGQPPKLLIYRASTLASGVPSRFSGSGSGTEYTLTISDLECADAATYYCQQDY
IYNDIDNAFGGGTEVVVKGDPVAPTVLIFPPAADQVATGTVTIVCVANKYFPDVTVTW
EVDGTTQTTGIENSKTPQNSADCTYNLSSTLTLTSTQYNSHKEYTCKVTQGTTSVVQS
FNRGDC

TABLE C
Heavy Chain CDRs

| | CDR- | | | CDR- | | | CDR- | | |
|---|---|---|---|---|---|---|---|---|---|
| Clone | H1 | Residues | Length | H2 | Residues | Length | H3 | Residues | Length |
| 1B7 | 49- | DYAM | 5 | 68- | IISSSGNTHYAR | 16 | 115- | NYPGYAN | 10 |
| | 53 | G (SEQ | | 83 | WARG (SEQ ID | | 124 | YAL (SEQ | |
| | | ID | | | NO: 12) | | | ID NO: 13) | |
| | | NO: 11) | | | | | | | |

TABLE D
Light Chain CDRs

| | CDR- | | | CDR- | | | CDR- | | |
|---|---|---|---|---|---|---|---|---|---|
| Clone | L1 | Residues | Length | H2 | Residues | Length | H3 | Residues | Length |
| 1B7 | 47- | QASESISNW | 11 | 73- | RASTL | 7 | 112- | QQDYIYNDI | 12 |
| | 57 | LS (SEQ ID | | 79 | AS | | 123 | DNA (SEQ ID | |
| | | NO: 14) | | | (SEQ ID | | | NO: 16) | |
| | | | | | NO: 15) | | | | |

CDR identification method per E. Kabat, T. Wu, H. Perry, Sequences of Proteins of Immunological Interest, fifth ed., U.S. Department of Health and Human Services, National Institutes of Health, Bethesda MD, 1992.

A polypeptide variant, antibody variant or variant CDR differs by about, for example, 1, 2, 3, 4, 5, 6, 7, 8, 9, 10, 20, 30, 40, 50, 60 or more amino acid residues (e.g., amino acid additions, substitutions or deletions) from a polypeptide shown in SEQ ID NOs: 1-16 or a fragment thereof. Where this comparison requires alignment, the sequences are aligned for maximum homology. The site of variation can occur anywhere in the polypeptide. In one embodiment of the invention a variant polypeptide has activity substantially similar to a polypeptide shown in SEQ ID NOs: 1-16. Activity substantially similar means that when the polypeptide is used to construct an antibody, the antibody has the same or substantially the same activity/binding as the wild-type antibody.

As used herein, percent identity of two amino acid sequences (or of two nucleic acid sequences) is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al. (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the invention.

Identity or identical means amino acid sequence (or nucleic acid sequence) similarity and has an art recognized meaning. Sequences with identity share identical or similar amino acids (or nucleic acids). Sequence identity is the percentage of amino acids identical to those in the antibody's original amino acid sequence, determined after the sequences are aligned and gaps are appropriately introduced to maximize the sequence identity as necessary. Thus, a candidate sequence sharing 85% amino acid sequence identity with a reference sequence requires that, following alignment of the candidate sequence with the reference sequence, 85% of the amino acids in the candidate sequence are identical to the corresponding amino acids in the reference sequence, and/or constitute conservative amino acid changes.

The invention also includes polypeptide variants or CDR variants of SEQ ID NOs: 1-16. Polypeptide variants or CDR variants of SEQ ID NOs: 1-16 can comprise one or more amino acid substitutions, additions or deletions. In one embodiment, a variant polypeptide or variant CDR includes an amino acid sequence at least about 75% identical to a sequence shown as SEQ ID NOs: 1-16. In one embodiment, the variant polypeptide or CDR is at least about 75%, 80%, 85%, 90%, 95%, 98%, 99%, 99.5% or more identical to SEQ ID NOs: 1-16. Variant polypeptides or variant CDRs encode a variant antibody, which is an antibody comprising an amino acid sequence of SEQ ID NOs: 1-16 in which one or more amino acid residues have been added, substituted or deleted. For example, the variable region of an antibody can be modified to improve its biological properties, such as antigen binding. Such modifications can be achieved by e.g., site-directed mutagenesis, PCR-based mutagenesis, cassette mutagenesis. Variant antibodies comprise an amino acid sequence which is at least about 75%, 80%, 85%, 90%, 95%, 98%, 99%, 99.5% or more identical to the amino acid sequence of a heavy or light chain variable region of SEQ ID NOs: 1-16.

Methods of introducing a mutation into an amino acid sequence are well known to those skilled in the art. See, e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989)). Mutations can also be introduced using commercially available kits such as “QuikChange™ Site-Directed Mutagenesis Kit” (Stratagene). The generation of a functionally active variant polypeptide by replacing an amino acid that does not influence the function of a polypeptide can be accomplished by one skilled in the art.

The variant polypeptides can have conservative amino acid substitutions at one or more predicted non-essential amino acid residues. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A polypeptide or antibody of the invention can be covalently or non-covalently linked to an amino acid sequence to which the polypeptide or antibody is not normally associated with in nature. Additionally, a polypeptide or antibody of the invention can be covalently or non-covalently linked to compounds or molecules other than amino acids. For example, a polypeptide or antibody can be linked to an indicator reagent (indicator reagents can include chromogenic agents, catalysts, such as enzyme conjugates, fluorescent compounds, such as fluorescein and rhodamine, chemiluminescent compounds, such as dioxetanes, acridiniums, phenanthridiniums, ruthenium, and luminol, radioactive elements, direct visual labels, as well as cofactors, inhibitors, magnetic particles, and the like; examples of enzyme conjugates include alkaline phosphatase, horseradish peroxidase, beta-galactosidase, and the like), an amino acid spacer, an amino acid linker, a signal sequence, a stop transfer sequence, a transmembrane domain, a protein purification ligand (e.g., glutathione-S-transferase, histidine tag, and staphylococcal protein A), or a combination thereof. In one embodiment of the invention a protein purification ligand can be one or more C amino acid residues at, for example, the amino terminus or carboxy terminus of a polypeptide of the invention. An amino acid spacer is a sequence of amino acids that are not usually associated with a polypeptide or antibody of the invention in nature. An amino acid spacer can comprise about 1, 5, 10, 20, 100, or 1,000 amino acids.

A polypeptide of the invention can be isolated from cells or tissue sources using standard protein purification techniques. Polypeptides of the invention can also be synthesized chemically or produced by recombinant DNA techniques. For example, a polypeptide of the invention can be synthesized using conventional peptide synthesizers.

A polypeptide of the invention can be produced recombinantly. A polynucleotide encoding a polypeptide of the invention can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system.

### Binding

Antibodies/binding portions thereof (antigen binding fragments) of the invention specifically bind HER2 (e.g., human HER2). “Specifically binds” means that the antibody recognizes and binds to HER2 with greater affinity than to other, non-specific molecules that are not HER2. For example, an antibody raised against an antigen (polypeptide) to which it binds more efficiently than to a non-specific antigen (e.g., a protein that is not related to or homologous to HER2) can be described as specifically binding to the antigen. Binding specificity can be tested using, for example, an enzyme-linked immunosorbant assay (ELISA), a radioimmunoassay (RIA), or a western blot assay using methodology well known in the art.

### Methods of Making Antibodies

Antibodies of the invention can be produced using methods known to those of skill in the art. For example, an HER2 antigen or a fragment thereof can be used to immunize animals, including rabbit. HER2 or a fragment thereof can be conjugated to a carrier protein and/or administered to the animals with an adjuvant. An HER2 antigen can comprise one or more epitopes (i.e., antigenic determinants). An epitope can be a linear epitope, sequential epitope or a conformational epitope. Epitopes within a polypeptide of the invention can be identified by several methods. See, e.g., U.S. Pat. No. 4,554,101; Jameson & Wolf, CABIOS 4:181-186 (1988). For example, HER2 can be isolated and screened. A series of short peptides, which together span the entire HER2 polypeptide sequence, can be prepared by proteolytic cleavage. By starting with, for example, 100-mer polypeptide fragments, each fragment can be tested for the presence of epitopes recognized in an ELISA. For example, in an ELISA assay an HER2 antigen, such as a 100-mer polypeptide fragment, is attached to a solid support, such as the wells of a plastic multi-well plate. A population of antibodies are labeled, added to the solid support and allowed to bind to the unlabeled antigen, under conditions where non-specific absorption is blocked, and any unbound antibody and other proteins are washed away. Antibody binding is detected by, for example, a reaction that converts a colorless substrate into a colored reaction product. Progressively smaller and overlapping fragments can then be tested from an identified 100-mer to map the epitope of interest.

Methods for preparing monoclonal antibodies from hybridomas are well known to those of skill in the art and include, e.g., standard cell culture methods and ascites production methods. Recombinant antibodies or fragments thereof produced by gene engineering can be made using the polynucleotide sequences of the invention. Genes encoding antibodies or fragments thereof can be isolated from hybridomas of the invention or other hybridomas. The genes can be inserted into an appropriate vector and introduced into a host cell. See, e.g., Borrebaeck & Larrick, Therapeutic Monoclonal Antibodies, Macmillan Publ. Ltd, 1990.

In one aspect, highly specific monoclonal antibodies were developed by immunizing rabbits, selecting spleenocytes and constructing commercial quantities of monoclonal antibodies suitable for clinical use. Most recombinant rabbit monoclonal antibodies are used only in research, so the use of monoclonal rabbit antibodies in the clinical space is unique. In addition, the antibodies described herein are superior for a number of reasons. For example, recombinant rabbit mAbs exhibit higher binding affinity to their ligand relative to recombinant mouse mAbs and, thereby, provide more reproducible results. Further, the rabbit monoclonal antibodies provided herein show limited/moderate to no therapeutic drug interference in the immunoassay.

Rabbit monoclonal antibodies (mAb) have been recognized for their advantages as research and diagnostic reagents: they have affinities 10-100 times higher than mouse mAbs; superior specificity that can distinguish even single amino acid differences and reduce cross-reactivity; broad epitope recognition that increases mAb diversity; great stability for consistent performance; and longer shelf life due to extra disulfide bonds in rabbit IgG (Feng L. et al. Am J Transl Res. 2011; 3 (3): 269-74; Rossi S. et al. American Journal of Clinical Pathology. 2005; 124 (2): 295-302; Vilches-Moure J G et al. J Vet Diagn Invest. 2005; 17 (4): 346-50).

To effectively discover specific mAbs, one can use methods available to the art, such as the single B cell based SMab™ platform for efficient high-throughput screening for specific rabbit mAbs of interest. Briefly, one first enriches and sorts protein-recognizing B cells from splenocytes using fluorescence activated cell sorting (FACS); sorted cells are cultured and stimulated in 1 cell/well; positive clones are identified from single B cells using enzyme-linked immunosorbent assays (ELISA) and other desired assays against the protein of interest; supernatants and RNA are collected for future analysis; genes of naturally paired IgG light and heavy chains are then cloned from positive clones; and then one expresses and validates selected mAb clones. SMab™ platform routinely generates 300-500 testable clones of mAbs in 3-4.5 months, about 30% to 50% faster than traditional hybridoma and display platforms. Use of a large pool of splenocytes and scalable high-throughput design increases the diversity of the initial mAb pool recognizing the protein of interest. SMab™ platform delivers the earliest functional characterization of protein-specific mAbs using the culture supernatants from intermediate steps to reduce antibody development time by removing unnecessary workload.

### Conjugates

Antibodies of the invention can be covalently attached to other molecules such that covalent attachment does not affect the ability of the antibody to bind to HER2. For example, antibodies can be modified by, e.g., glycosylation, acetylation, pegylation, phosphorylation, amidation, derivatization by known protecting/blocking groups (e.g., methyl group, ethyl group, carbohydrate group), proteolytic cleavage, linkage to a cellular ligand or other protein.

Conjugated antibodies can be bound to various molecules including, for example, polymers, hyaluronic acid, fluorescent substances, luminescent substances, haptens, enzymes, metal chelates, cytotoxic agents, radionuclides, and drugs.

### Methods of Detection

One embodiment of the invention provides methods of detecting HER2 polypeptides in a sample. The methods comprise contacting the sample suspected of containing HER2 polypeptides with an antibody or antigen binding portion thereof of the invention to form HER2/antibody complexes. The presence of the HER2/antibody complexes are detected, thereby detecting the presence of the HER2 polypeptides. In some embodiments, two different antibodies or antigen binding portion thereof of the invention are used in detection of HER2 (such as antibodies comprising SEQ ID NOs: 5-10 and also contacting with antibodies comprising SEQ ID NOs: 11-16; capture antibody and label antibody, include 1C5 and 1B7).

The test sample can be, e.g., lymph node or tissue aspirate, serum, whole blood, plasma, circulating tumor cells, tumor cells or tissue (e.g., tissue biopsy) or ascites fluid. Polypeptide/antibody complexes can be detected by any method known in the art, including, but not limited to, enzyme-linked immunosorbent assay (ELISA), multiplex fluorescent immunoassay (MFI or MFIA), radioimmunoassay (RIA), sandwich assay, western blotting, immunoblotting analysis, an immunohistochemistry method, immunofluorescence assay, fluorescence-activated cell sorting (FACS) or a combination thereof.

An immunoassay for HER2 can utilize one antibody or several different antibodies. Immunoassay protocols can be based upon, for example, competition, direct reaction, or sandwich type assays using, for example, labeled antibody. Antibodies of the invention can be labeled with any type of label known in the art, including, for example, fluorescent, chemiluminescent, radioactive, enzyme, colloidal metal, radioisotope and bioluminescent labels.

Antibodies of the invention or antigen-binding portions thereof can be bound to a support and used to detect the presence of HER2. Supports include, for example, glass, polystyrene, polypropylene, polyethylene, dextran, nylon, amylases, natural and modified celluloses, polyacrylamides, agaroses and magletite.

Antibodies of the invention can be used in a method of the diagnosis of a hyperproliferative disorder by obtaining a test sample from, e.g., a human or animal suspected of having a hyperproliferative disorder. The test sample is contacted with antibodies or antigen-binding portions thereof of the invention under conditions enabling the formation of antibody-antigen complexes (i.e., immunocomplexes). One of skill in the art is aware of conditions that enable and are appropriate for formation of antigen/antibody complexes. The amount of antibody-antigen complexes (including, for example, a complex of an antibody or antigen-binding portion thereof and HER2) can be determined by methodology known in the art. A level that is higher than that formed in a control sample indicates the presence of a hyperproliferative disorder. The amount of antibody/antigen complexes can be determined by methods known in the art.

A HER2 positive hyperproliferative disorder can be a neoplastic disorder including breast cancer, ovarian cancer, pancreatic cancer, bladder cancer, adenocarcinoma of the lung, uterine cancer (such as uterine serous endometrial carcinoma), gastric cancer, esophageal cancer, colon cancer, and head and/or neck cancers and/or salivary duct carcinoma.

The antibodies/assays described herein can be used to identify and monitor patients having tumors that overexpress HER2 and, thus, are candidates for targeted drug treatment. The antibodies/assays described herein show limited to no interference with therapeutic agents. Accordingly, the immunoassays described herein fill an unmet need in the breast cancer care space.

The immunoassays described herein can be used to test for HER2 positive breast cancer, monitor the serum levels of HER2 in patients receiving drug therapy, to detect recurrence, or detect HER2 disease in women that tested tissue HER2 negative. The immunoassays described herein can be used to detect an elevated or rising level of serum HER2 in a woman, which can indicate the appearance of HER2 disease in women that were thought to be HER2-negative (e.g., by tissue testing). The immunoassays described herein also can be used in conjunction with measuring circulating tumor cells (CTC) or as an adjunct to identify, or help identify, patients that are in need of, or would benefit from, a positron emission tomography (PET) scan.

### Lateral Flow Assays (LFAs)

A lateral flow assay (LFA) is based on the movement of a liquid sample though a polymeric strip with attached molecules that interact with the analyte, providing a signal that can be visually detected. LFA is generally a paper-based platform for the detection and/or quantification of analytes (such as proteins, haptens, nucleic acids and amplicons) in what are often complex mixtures, where the sample is placed on a test device and the results are displayed within about 5-30 min, such 5-10 minutes. Low development costs and ease of production of LFAs have resulted in the expansion of its applications to multiple fields in which rapid tests are needed, such as biomedicine, agriculture, food and environmental sciences. LFA-based tests are widely used in hospitals, physician's offices and clinical laboratories for the qualitative and quantitative detection of specific antigens and antibodies, as well as products of gene amplification, in such settings as veterinary medicine, quality control, product safety in food production, and environmental health and safety, including to screen for animal and human diseases, pathogens, chemicals, toxins and water pollutants, among others.

For LFA, a liquid sample (such as urine, saliva, sweat, serum, plasma, whole blood and other fluids) containing the analyte of interest moves without the assistance of external forces (capillary action) through various zones of polymeric strips, on which molecules that can interact with the analyte are attached. A typical lateral flow test strip can consist of overlapping membranes that are mounted on a backing card for better stability. The sample is applied at one end of the strip, on the adsorbent sample pad, which can be loaded with buffer salts and surfactants that make the sample suitable for interaction with the detection system. The sample migrates through the conjugate release pad, which contains antibodies that are specific to the target analyte and are conjugated to colored or fluorescent particles—such as colloidal gold and latex microspheres (depending on the elements of recognition used, LFAs can be categorized into different types, such as ‘lateral flow immunoassays’ (LFIAs), in which antibodies are used as recognition elements, and nucleic acid LFA (NALFA), in which the detection of amplicons which can be formed during the polymerase chain reaction (PCR) are used). The sample, together with the conjugated antibody bound to the target analyte, migrates along the strip into the detection zone. This is generally a porous membrane (usually composed of nitrocellulose) with specific biological components (mostly antibodies or antigens) immobilized in lines. Their role is to react with the analyte bound to the conjugated antibody. Recognition of the sample analyte results in an appropriate response on the test line, while a response on the control line indicates the proper liquid flow through the strip. The read-out, represented by the lines appearing with different intensities, can be assessed by eye or using a dedicated reader (device).

Provided herein is a point-of-care multiple diagnostic assay with multiple test lines allowing the rapid and simultaneous detection of multiple analytes present in samples, including, for example, HER2 positive hyperproliferative disorders such as a neoplastic disorder including breast cancer, ovarian, stomach, adenocarcinoma of the lung, uterine cancer (such as uterine serous endometrial carcinoma), gastric cancer and/or salivary duct carcinoma, providing a powerful toll for cancer detection and progression, for example, before, after and/or during treatment. In order to test multiple analytes simultaneously under the same conditions, additional test lines of antibodies specific to different analytes can be immobilized in an array format. On the other hand, multiple test lines loaded with the same antibody can be used for semi-quantitative assays. The principle of this ‘ladder bars’ assay is based on the stepwise capture of colorimetric conjugate-antigen complexes by the immobilized antibody on each successive line, where the number of lines appearing on the strip is directly proportional to the concentration of the analyte. The liquid flows across the device because of the capillary force of the strip material and, to maintain this movement, an absorbent pad can be attached at the end of the strip. The role of the absorbent pad is to wick the excess reagents and prevent backflow of the liquid. A current example of an LFA is a pregnancy test stick.

Two formats of the LFIA can be distinguished: direct and competitive. A direct test is used for larger analytes such as the p24 antigen used in the human immunodeficiency virus (HIV) test as well as analytes with multiple antigenic sites such as human chorionic gonadotropin (hCG) used in pregnancy tests. The hCG test is an example of a sandwich-based assay, where the target is immobilized between two complementary antibodies. In the direct test, the presence of the test line indicates a positive result and the control line usually contains species-specific anti-immunoglobulin antibodies, specific for the antibody in the particular conjugate. In the case of small molecules with single antigenic determinants, which cannot bind to two antibodies simultaneously, competitive tests are used. In this type of test, the analyte blocks the binding sites on the antibodies on the test line, preventing their interactions with the colored conjugate. Therefore, a positive result is indicated by the lack of signal in the test line, while the control line should be visible independently of the test result.

As for a label colloidal gold is a widely used label in commercial LFIA. Another popular label is latex, which can be tagged with a variety of detector reagents such as colored or fluorescent dyes, and magnetic or paramagnetic components. As latex can be produced in multiple colors, it has an application in multiplex assays, which require discrimination between numerous lines. Carbon and fluorescent labels, or enzymatic modification of the labels, are also used. Carbon nanotubes, fluorescent labels, quantum dots, upconverting phosphors can all be used as labels. Another detection system that can be used is FACTT, an acronym for a sensitive protein detection system whereby amplification of the detection mAb occurs when coupled with T7 polymerase. Rather than measuring the mAb directly, the reader detects RNA molecules generated by the polymerase, thus greatly amplifying the result. This test can result in a qualitative color change but may also benefit from a reader (device). It may take 20-30 minutes.

There are many advantageous to using such an assay including, for example, point-of-care, providing inexpensive, rapid and easy tests desirable in many industries/countries and because of their long shelf life and the fact that refrigeration is often not required for storage, these tests are well suited for use in developing countries, small ambulatory care settings, remote regions and battlefields. Further as the visual result is usually clear, no additional equipment is needed; however, an optional device can be used for the readout.

The following examples are provided for exemplification purposes only and are not intended to limit the scope of the invention described in broad terms above.

### EXAMPLES

### Example I

### A. Linearity

A linearity assay was utilized to evaluate the reportable range. Two natural samples with suitable HER2 levels, 35SC and 40SC, were diluted 1:25, 1:100 and 1:200, in addition to the 1:50 dilution specified in the assay's standard operating procedure (SOP). After measurement, linearity was demonstrated by quantifying these values as a percentage of the expected HER2 concentration for each tested dilution. Ranges between 80% and 120% of the expected value indicate acceptable linearity.

Linearity was well within acceptable tolerances for both natural samples tested. For the 1:25, 1:100 and 1:200 dilutions, serum 35SC gave corrected values of 98%, 101% and 105% of the expected concentration, respectively; and serum 40SC gave corrected values of 101%, 99% and 100%, respectively. See Table 1.

TABLE 1
1:50 Dilution of standards

| | | | Corrected | Percentage |
|---|---|---|---|---|
| | | Concentration | Concentration | of expected |
| Sample | Dilution | (ng/ml) | (ng/ml) | dilution |
| 35SC | 1:25 | 18.143 | 9.0715 | 98% |
| | 1:50 (SOP) | 9.289 | 9.289 | 100% |
| | 1:100 | 4.698 | 9.396 | 101% |
| | 1:200 | 2.443 | 9.772 | 105% |
| 40SC | 1:25 | 27.706 | 13.853 | 101% |
| | 1:50 (SOP) | 13.687 | 13.687 | 100% |
| | 1:100 | 6.785 | 13.57 | 99% |
| | 1:200 | 3.407 | 13.628 | 100% |

### B. Cross-Reactivity

For cross-reactivity, the following four recombinant human (rh) proteins (three family members related to HER2 and one unrelated protein) were tested individually at the elevated concentration of 200 ng/ml in the assay alongside the standard curve: rhEGFR, rhHER3, rhHER4, and rhPD-L1 (each provided by Sino Biological). Cross-reactivity was assessed by calculating the percentage of measured recombinant protein concentration versus the loaded initial concentration of 200 ng/mL. Any recombinant protein found to generate values greater than 5.0% of that expected for HER2 was considered cross-reacting.

None of the four recombinant proteins tested cross-reacted in the HER2 ELISA at 200 ng/ml, meeting acceptability requirements. See Table 2.

TABLE 2
1:50 Dilution of standards
Cross Reactivity

| EGFR Family | Concentration | % Cross |
|---|---|---|
| Member | (ng/ml) | Reactivity |
| EGFR (200 ng/ml) | 0.197 | 0.10% |
| HER3 (200 ng/ml) | 0.206 | 0.10% |
| HER4 (200 ng/ml) | 0.1 | 0.05% |
| PD-L1 (200 ng/ml) | 0.066 | 0.03% |

### C. Protein Interference

To determine interference with related family proteins, EGFR, HER3 and HER4 at the same elevated concentration of 200 ng/ml were individually added to a midpoint rhHER2 concentration of approximately 7 ng/ml (considered the reference sample). Following the assay run, the measured HER2 concentration was compared to the expected concentration, and percent recovery was calculated. Any protein that, when present, generated a measured concentration less than 80% or greater than 120% of that expected for HER2 was considered interfering.

Recovery percentages were 96.7% for rhEGFR, 99.7% for rhHER3, and 109.6% for rhHER4. Therefore, these related recombinant proteins showed no interference with the HER2 ELISA at 200 ng/mL, and results met acceptability requirements. See Table 3.

TABLE 3
1:50 Dilution of standards
Interference

| | | % of |
|---|---|---|
| | Concentration | Reference |
| EGFR Family Member | (ng/ml) | Sample |
| HER2 Reference Sample | 6.8 | 100.0% |
| HER2 + EGFR (200 ng/ml) | 6.573 | 96.7% |
| HER2 + HER3 (200 ng/ml) | 6.78 | 99.7% |
| HER2 + HER4 (200 ng/ml) | 7.45 | 109.6% |

### D. Drug Interference

To determine interference with relevant drugs, the following three therapeutic antibodies were run for interference in serum samples: trastuzumab (Herceptin; Roche), pertuzumab (Perjeta; Roche), and pembrolizumab (Keytruda; Merck). Each drug was spiked at the physiologically relevant concentration of 100 μg/mL into endogenous samples with a known assay measurement (a natural reference point). Any changes from the expected concentration were determined and represented as percent interference. A therapeutic antibody that, when present, generated a change in measured concentration of greater than 10% of that expected for HER2 was considered interfering.

Using two samples, B54 and 20SC, average interference percentages were 6.13% for Herceptin and 3.72% for Perjeta. Using eight seras and two experimental dates to confirm results, the average interference percentage was 4.5% for Perjeta. Therefore, all three antibody drugs tested, Herceptin, Keytruda, and Perjeta, exhibited no interference with the HER2 ELISA at 100 μg/mL. See Table 4.

TABLE 4

| | | Sample | | Avg. % |
|---|---|---|---|---|
| | Drug | % | Interference | Interference |
| 1:50 Dilution | Herceptin | B54 | 6.59% | 6.13% |
| of standards | | 20SC | 5.66% | |
| and samples | Keytruda | B54 | 5.92% | 3.72% |
| | | 20SC | 1.52% | |
| | Perjeta | 16SC | 4.1% | 4.5% |
| | | 27SC | 4.1% | |
| | | 34SC | 10.6% | |
| | | 43SC | 8.1% | |
| | | 28SC | 1.5% | |
| | | 35SC | 0.5% | |
| | | 37SC | 5.0% | |
| | | 42SC | 2.0% | |

All publications, nucleotide and amino acid sequence identified by their accession nos., patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of nucleic acids or polypeptides or a series of nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

1. An anti-HER2 monoclonal antibody or binding fragment thereof comprising a heavy chain variable domain (VH) comprising the amino acid sequence of SEQ ID NO: 1 or at least 95% identity thereto and a light chain variable domain (VL) comprising the amino acid sequence of SEQ ID NO:2 or at least 95% identity thereto.

2. An anti-HER2 monoclonal antibody or binding fragment thereof comprising a heavy chain and a light chain, wherein: (i) the heavy chain comprises three CDR regions having the amino acid sequence SEQ ID NO: 5, 6, 7 or at least 95% identity thereto; and (ii) the light chain comprises three CDR regions having the amino acid sequence SEQ ID NO: 8, 9, 10 or at least 95% identity thereto.

3. The anti-HER2 monoclonal antibody or binding fragment thereof of claim 1, wherein the antibody is conjugated to a detection agent.

4. A composition comprising the anti-HER2 antibody of claim 1 and a carrier.

5. A method to detect HER2 polypeptides or fragments thereof in a test sample comprising:
a) contacting the anti-HER2 monoclonal antibody or binding fragment thereof of claim 1 with a test sample under conditions that allow polypeptide/antibody complexes to form; and
b) detecting polypeptide/antibody complexes of a), wherein the detection of polypeptide/antibody complexes is an indication that the HER2 polypeptide is present in the sample.

6. A method to monitor HER2 polypeptides or fragments thereof in a sample from a subject comprising:
a) contacting at least one anti-HER2 monoclonal antibodies or binding fragments thereof of claim 1 with said sample under conditions that allow polypeptide/antibody complexes to form;
b) detecting polypeptide/antibody complexes of a), wherein the detection of polypeptide/antibody complexes indicates HER2 polypeptides or fragments thereof are present in the subject; and
c) performing steps a) and b) at a plurality of time points so as monitor HER2 polypeptides or fragments thereof in said subject over time.

7. The method of claim 5, further comprising contacting the sample of a) with a second anti-HER2 antibody or fragment thereof comprising a heavy chain variable domain (VH) comprising the amino acid sequence of SEQ ID NO: 3 or at least 95% identity, a light chain variable domain (VL) comprising the amino acid sequence of SEQ ID NO: 4 or at least 95% identity or an anti-HER2 antibody or fragment thereof comprising a heavy chain and a light chain, wherein (i) the heavy chain comprises three CDR regions having the amino acid sequence SEQ ID NO: 11, 12, 13 or at least 95% identity; and (ii) the light chain comprises three CDR regions having the amino acid sequence SEQ ID NO: 14, 15, 16 or at least 95% identity.

8. The method of claim 5, wherein the sample is contacted in a) with:
i) a capture antibody or a binding fragment thereof, and
ii) a detection antibody or a binding fragment thereof.

9. The method of claim 8, wherein the capture and detection antibodies bind to HER2.

10. The method of claim 8, wherein the capture antibody is immobilized.

11. The method of claim 8, wherein the detection antibody comprises a detection agent.

12. The method of claim 6, wherein the subject is being treated with a therapeutic agent.

13. The method of claim 12, wherein the therapeutic agent is trastuzumab, trastuzumab emtansine, pembrolizumab, pertuzumab, nivolumab, atezolizumab or a combination thereof.

14. The method of claim 6, wherein the subject is being treated with trastuzumab, trastuzumab emtansine, pembrolizumab, pertuzumab, nivolumab, atezolizumab or a combination thereof, wherein the trastuzumab, trastuzumab emtansine, pembrolizumab, pertuzumab, nivolumab, atezolizumab or a combination thereof does not interfere or only moderately interferes with the binding of the capture and/or detection antibodies or binding fragments thereof.

15. The method of claim 5, wherein the sample is lymph node or tissue aspirate (e.g., breast), serum, whole blood, plasma, urine, saliva, tears, cerebrospinal fluid, supernatant from normal cell lysates, supernatant from pre-neoplastic cell lysates, supernatant from neoplastic cell lysates and/or supernatants from carcinoma cell lines maintained in tissue culture.

16. The method of claim 5, wherein the detection of b) is carried out with the use of a lateral flow assay.
